(19) United States
(12) Patent Application Publication
(10) Pub. No.: US 2021/0308245 A1
(43) Pub. Date: Oct. 7, 2021

(54) CONSTRUCTION AND USE OF TANDEM EPITOPE POLYPEPTIDE OF OUTER MEMBRANE PROTEIN OF CAMPYLOBACTER JEJUNI, GENE, RECOMBINANT PLASMID AND RECOMBINANT BACTERIA

(71) Applicants: JINHUA POLYTECHNIC, Jinhua City (CN); HANGZHOU MEDICAL COLLEGE, Hangzhou City (CN)

(72) Inventors: Hongqiang LOU, Jinhua City (CN); Xiusheng SHENG, Jinhua City (CN); Haohao CHEN, Jinhua City (CN); Aihua SUN, Jinhua City (CN); Shaoye WAN, Jinhua City (CN); Shuiqin FANG, Jinhua City (CN); Xusheng LI, Jinhua City (CN)

(73) Assignees: JINHUA POLYTECHNIC, Jinhua City (CN); HANGZHOU MEDICAL COLLEGE, Hangzhou City (CN)

(21) Appl. No.: 17/220,355

(22) Filed: Apr. 1, 2021

(30) Foreign Application Priority Data
Apr. 3, 2020 (CN) 202010259854.7

(51) Int. Cl. A61K 39/02; C07K 14/205; C12N 15/66; A61P 31/04; C12N 15/70

(52) U.S. Cl. CPC A61K 39/0208; C07K 14/205; C12N 15/70; A61P 31/04; C12N 15/66

(57) ABSTRACT

The disclosure provides the construction and use of tandem epitope polypeptide of outer membrane protein of campylobacter jejuni, gene, recombinant, plasmid recombinant bacteria, belonging to the technical field of genetic engineering vaccines. The tandem epitope polypeptide of outer membrane protein of campylobacter jejuni can activate the immune response of mice, and has the effect of efficiently preventing Campylobacter jejuni colonization. The tandem epitope polypeptide of the disclosure can be used for preparing vaccines for preventing campylobacter jejuni infection.

### CROSS REFERENCE TO RELATED APPLICATION

This patent application claims the benefit and priority of Chinese Patent Application No. 202010259854.7 filed on Apr. 03, 2020, the disclosure of which is incorporated by reference herein in its entirety as part of the present application.

### TECHNICAL FIELD

The present disclosure relates to the technical field of genetic engineering vaccines, in particular to the construction and use of tandem epitope polypeptide of outer membrane protein of Campylobacter jejuni, gene, recombinant plasmid and recombinant bacteria.

### BACKGROUND ART

Campylobacter jejuni is a gram-negative micro-aerobic zoonotic pathogen discovered in 1970s. It mainly infects humans by polluted food, thus causing acute enteritis, and also causing Guillain-Barra syndrome (GBS), reactive arthritis and Reiter's syndrome.

Vaccination is an effective way to prevent infectious diseases. For example, the research of Helicobacter pylori vaccine has made great progress, while Campylobacter jejuni vaccine research has not made more progress due to the differences in molecular structure of different serotypes or potential peripheral nerve immune damage. Multiple antigenic peptides (MAP) vaccine is a new generation of genetic engineering vaccine, which is artificially cross-linked by multiple dominant epitopes identified by Reverse Vaccinology technology and polymer core carrier. MAP vaccine has many advantages, such as presenting multiple antigenic epitopes at the same time, having strong antigen presenting effect, non-covalent binding between various antigenic peptide branches to form constitutive epitope to improve immune effect, and individual amino acid mutation does not affect antigen presenting and subsequent immune response. The length of antigen epitope is usually less than 30 amino acids, wherein the predominant epitope can usually reflect more than 50% immunogenicity of the finished protein antigen molecule. In addition, the epitope short peptide can be expressed repeatedly in tandem to significantly improve its yield. However, there is still no research report on MAP genetic engineering vaccine of Campylobacter jejuni. With the increasing of drug-resistant strains of Campylobacter jejuni, it is of great practical significance to seek a new, safe and efficient subunit vaccine of Campylobacter jejuni.

### SUMMARY

The purpose of the present disclosure is to provide construction and use of tandem epitope polypeptide of outer membrane protein of Campylobacter jejuni gene, recombinant plasmid, recombinant bacteria. The epitope polypeptide of Campylobacter jejuni outer membrane protein can induce immune response and reduce the number of Campylobacter jejuni colonization.

In order to achieve the above purpose, the present disclosure provides the following technical solutions:

The disclosure provides a tandem epitope polypeptide of outer membrane protein of Campylobacter jejuni, the amino acid sequence of which is set forth in SEQ ID NO: 1

The disclosure also provides a gene encoding the tandem epitope polypeptide of of outer membrane protein of Campylobacter jejuni described in the above solution, the nucleotide sequence of which is set forth in SEQ ID NO: 2

The disclosure also provides a recombinant plasmid comprising the gene described in the above solution.

In some embodiments, the recombinant plasmid is obtained by inserting the gene into pET30a as a original plasmid.

In some embodiments, the gene is inserted between Nde I and Hind III restriction sites on pET30a.

The disclosure also provides a recombinant bacteria comprising the recombinant plasmid described in the above solution.

The disclosure also provides use of the tandem epitope polypeptide of outer membrane protein of Campylobacter jejuni, the gene, the recombinant plasmid or recombinant bacteria described in the above solution in preparing vaccine for preventing Campylobacter jejuni infection.

Beneficial effects: the disclosure provides construction and use of tandem epitope polypeptide of outer membrane protein of Campylobacter jejuni, gene, recombinant plasmid, recombinant bacteria. The tandem epitope polypeptide of outer membrane protein of Campylobacter jejuni can activate the immune response of mice, and has the effect of preventing Campylobacter colonization with high efficiency. The tandem epitope polypeptide of outer membrane protein of Campylobacter jejuni can be used for preparing vaccines for preventing Campylobacter jejuni infection.

### BRIEF DESCRIPTION OF THE DRAWINGS

FIG. 1 is a structural schematic diagram of recombinant prokaryotic expression vector pET30a;

FIG. 2 shows the double digestion results of Nde I and Hind III after connecting Omp18 gene of Campylobacter jejuni with pET30a;

FIG. 3 shows the double digestion results of Nde I and Hind III after connecting AhpC gene of Campylobacter jejuni with pET30a;

FIG. 4 shows the double digestion results of Nde I and Hind III after connecting FlgH gene of Campylobacter jejuni with pET30a;

FIG. 5 shows the purification effect of rOmp18, rAhpC and rFlgH and their binding with corresponding antibodies; wherein A shows the coomassie brilliant blue staining result after purification by rOmp18, rAhpC and rFlgH affinity chromatography. B shows the Western Blot detection of rOmp18, rAhpC and rFlgH after binding with corresponding antibodies;

FIG. 6 shows the binding reactivity of Omp18, AhpC and FlgH T-B combined antigenic epitopes with corresponding antibodies;

FIG. 7 shows the nucleotide sequencing result of rAhpC-2/Omp18-1/FlgH-1 tandem epitope peptide, wherein red, blue and yellow represent the nucleotide sequence of AhpC-2, Omp18-1 and FlgH-1T-B combined epitope respectively;

FIG. 8 shows the amino acid sequence of rAhpC-2/Omp18-1/FlgH-1 tandem epitope peptide, wherein red, blue and yellow represent the amino acid sequence of AhpC-2, Omp18-1 and FlgH-1T-B combined epitope respectively;

FIG. 9 shows the detection results of tandem epitope peptide by SDS-PAGE (A) and Western Blot (B);

FIG. 10 shows the changes of serum specific IgGs levels in mice immunized with different recombinant epitope peptides, * indicates that compared with non-immunized mice, P<0.05;

FIG. 11 shows the change of CD4+cells.

FIG. 12 shows the change of CD4+IFN-γ+cells.

FIG. 13 shows the change of CD4+IL-4+cells.

FIG. 14 shows the change of CD8+cells.

FIG. 15 shows the change of IFN-γ+CD8+cells.

FIG. 16 shows the change of CD8+IL-4+cells.

FIG. 17 shows the weight changes of mice in each group after immunization;

FIG. 18 shows the pathological examination results of intestinal mucosa of immunized mice.

### DETAILED DESCRIPTION OF THE EMBODIMENTS

The present disclosure provides a tandem epitope polypeptide of outer membrane protein of Campylobacter jejuni, wherein the amino acid sequence of the tandem epitope polypeptide of outer membrane protein of Campylobacter jejuni (TKKAL or Campylobacter jejuni OMP18-1/AhpC-2/FlgH-1 tandem epitope polypeptide) is set forth in SEQ ID NO: 1, which is specifically as follows:

MTKKALDFTAPAVLGGSKAVKEALIAKGVNADRIAVKSYGETNPVCTEKT
KACDAQNRRGGSFGCSATVDPQISMKPPAYVEELAPKQSNNVESAPGSLF
GGGSTKKALDFTAPAVLGGSKAVKEALIAKGVNADRIAVKSYGETNPVCT
EKTKACDAQNRRGGSFGCSATVDPQISMKPPAYVEELAPKQSNNVESAPG
SLFGGGSTKKALDFTAPAVLGGSKAVKEALIAKGVNADRIAVKSYGETNP
VCTEKTKACDAQNRRGGSFGCSATVDPQISMKPPAYVEELAPKQSNNVES
APGSLFGHHHHHH.

The tandem epitope polypeptide of outer membrane protein of Campylobacter jejuni comprises three polypeptide epitopes, i.e. FlgH-1, OMP18-1 and AHPC-2; the amino acid sequence of the epitope FlgH-1 is set forth in SEQ ID NO: 3, specifically, FGC SATVDPQISMKPPAYVEELAPKQSNNVESAPGSLFG. The amino acid sequence of the epitope OMP18-1 is set forth in SEQ ID NO: 4, specifically: KAVKEALIAKGVNADRIAVKSYGETNPVCTEKTKACDAQNRR. The amino acid sequence of the epitope AhpC-2 is set forth in SEQ ID NO: 5, specifically: TKKALDFTAPAVLGNNEIVQD FNLYKNIGPKGAVVF; the FlgH-1, OMP18-1 and AhpC-2 are connected in tandem by a connecting peptide GGS and repeated twice.

The disclosure also provides a gene encoding tandem epitope polypeptide of outer membrane protein of Campylobacter jejuni described in the above solution, wherein the nucleotide sequence of the gene is set forth in SEQ ID NO: 2, which is specifically as follows:

catatgaccaaaaaagcgctggattttaccgcaccggcagttctgggcgg
tagtaaagccgttaaagaagcactgattgcgaaaggcgtt
aacgcagatcgtatcgcggttaaaagctacggcgaaaccaacccggtttg
caccgagaaaaccaaagcgtgcgacgcacaaaatcgc
cgcggcggtagttttggttgttctgcgaccgttgatccgcagatttccat
gaaaccgccggcatacgttgaagaactggcaccgaaacag
agcaacaacgttgaatctgcaccgggtagtctgtttggeggeggtagcac
taagaaagcactagactttacagcgccagctgtacttgga
ggcagcaaggcggtcaaggaggcgctgattgcgaaaggtgttaacgcgga
tcgtattgcggtcaaaagctacggcgaaaccaatccg
gtttgtaccgaaaaaaccaaagcctgcgacgctcaaaatcgccgtggcgg
tagttttggttgttctgcaaccgtagatccgcagattagca
tgaaaccgccggcatacgttgaagaactggcaccgaaacagagcaataac
gttgaaagcgcaccgggtagtctgtttggcggcggtag
taccaaaaaagcgctggattttaccgcaccggcagttctgggcggtagca
aagccgttaaagaagcgctgatcgcaaaaggcgttaacg
cagatcgtattgcggtcaaaagctacggcgaaaccaatccggtttgcacc
gaaaaaaccaaagcctgcgacgcacaaaatcgtcgcgg
agggagcttcggctgctcagcgactgtcgacccacaaatcagcatgaagc
cacctgcgtatgtagaggaactcgcgccaaagcaaagt
aataacgtagagagcgctccgggtagtctgtttggtcatcatcatcacca
tcactaatgaaagett;

The gene encoding tandem epitope polypeptide of outer membrane protein of Campylobacter jejuni described in the above solution is subjected to codon optimization treatment. The software for codon optimization is preferably MaxCodon™ Optimization program (V13).

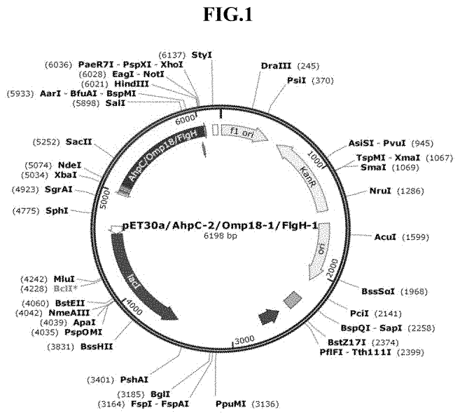

The disclosure also provides a recombinant plasmid containing the gene described in the above solution. Preferably, the recombinant plasmid is obtained by inserting the gene into pET30a as a original plasmid. Preferably, the gene is inserted between Nde I and Hind III restriction sites on pET30a. The structural diagram of the recombinant plasmid is shown in FIG. 1. In the present disclosure, the recombinant plasmid is constructed and obtained by Detai Biotechnology (Nanjing) Co., Ltd.

The disclosure also provides a recombinant bacteria containing the recombinant plasmid described in the above solution. Preferably, The recombinant bacteria is recombinant Escherichia coli. Preferably, The recombinant Escherichia coli comprises a recombinant Top10 clone strain and a recombinant BL21(DE3) expression strain. In the present disclosure, the recombinant bacteria is constructed and obtained by Detai Biotechnology (Nanjing) Co., Ltd.

The disclosure also provides the tandem epitope polypeptide of outer membrane protein of Campylobacter jejuni, the gene, the recombinant plasmid or the recombinant bacteria in preparing a vaccine for preventing Campylobacter jejuni infection. The tandem epitope polypeptide of outer membrane protein of Campylobacter jejuni is an effective epitope for Campylobacter jejuni MAP genetic engineering vaccine.

The disclosure also provides the tandem epitope polypeptide of outer membrane protein of Campylobacter jejuni that can inhibit the colonization of Campylobacter jejuni in intestinal tract and prevent infection caused by Campylobacter jejuni.

The technical solutions provided by the present disclosure will be described in detail with examples below, but they cannot be understood as limiting the scope of protection of the present disclosure.

### EXAMPLE 1

1 Experimental Method

1.1 Prediction of T-B combined Epitope in Outer Membrane Protein Omp18, AhpC and FlgH

The whole genome sequence of Campylobacter jejuni NCTC11168 strain was obtained from National Center for Biotechnology Information (http://www.ncbi.nlm.nih.gov/nuccore/AL111168.1). The gene sequences of Omp18, AhpC, FlgH and their products are from NC_002163.1, CAL34485.1 and RNF61095.1 respectively. The signal peptides and sequences of Omp18, AhpC and FlgH were predicted by Signalp4.1 (http://www.cbs.dtu.dk/services/SignalP/), and the transmembrane region was predicted by http://www.cbs.dtu.dk/services/TMHMM/, the epitope of b cells was predicted by IEDB (http://tools.iedb.org/bcell/) online software, and the epitope of T cells was predicted by http://www.syfpeithi.de/bin/mhcserver.dll/epigopeprediction.htm. According to the epitope scores of T and B cells and their overlap, the T-B combined epitope was predicted.

1. 2 Synthesis of 2T-B combined Epitope Peptide

The 2T-B combined epitope peptide was obtained by solid-phase polypeptide synthesis technique, which adopts the N-α-Fmoc protected amino acid as raw material and Fmoc-AA-Wang resin as carrier, and HBTU coupling method, and the purity was over 95% by HPLC.

1.3 Prokaryotic Expression System Construction and Antibody Preparation of Outer Membrane Protein Omp18, Ahpc and Flgh

1.3.1 Separation of Target Gene Fragment and Expression Vector by Enzyme Digestion and Electrophoresis

(1) Omp18, AhpC, FlgH gene cloning and expression vector plasmid pET30a were digested with restriction endonucleases Nde I and Hind III in 37° C. water bath for 2h. Enzyme digestion reaction system: Nde I 1 μL, Hind III 1 μL, 10× enzyme digestion buffer 2 μL, ultrapure water 5.5 μL, total volume 20 L.

(2) The enzyme digestion product was mixed with an equal volume of 2× electrophoresis sample loading buffer, and the mixed solution was added to the 1.5% agarose gel sample hole containing 1 μg/mL ethidium bromide, and electrophoresis was performed at 100V for 30 min.

1.3.2 Recovery of Target Gene Fragments and Vectors

(1) After electrophoresis, the target gene was observed on an ultraviolet lamp, the target strip was cut and put in a 1.5 mL centrifuge tube, and then added with 4 times the volume of Solution SN to mix well.

(2) The 3S column was put in the collecting tube, the mixed solution was transferred to the column, and placed at room temperature for 2 min, then covered with the centrifuge tube cover, and centrifuged at room temperature for 1 min at 10000 r/min.

(3) The waste liquid in the collecting tube was poured out, the 3S column was put in the same collecting tube, 600 μL Wash Solution was added, and the collecting tube was centrifuged at room temperature of 10000 r/min for 1 min.

(4) Step (3) was repeated once.

(5) The waste liquid in the collecting tube was poured out, the 3S column was put in the same collecting tube, and the collecting tube was centrifuged at room temperature of 10000 r/min for 2 min.

(6) The 3S column was put into a new 1.5 mL centrifuge tube, 30 μL TE buffer was added in the center of the 3S column membrane, the tube was placed at room temperature for 2 min, and centrifuged at room temperature for 1 min at 10000 r/min.

(7) The collected liquid included the recovered DNA fragment, which could be used immediately or stored at −20° C. for later use.

1.3.3 Connection of Target Fragments

The recovered target fragment and linearized vector pET30a were ligated in the ligation reaction fluid in a microtube, and the product was treated overnight at 16° C. in a PCR amplifier. The connection reaction solution is shown in Table 1:

TABLE 1
Connection system

| Components | Volume | |
|---|---|---|
| 10 × T4 DNA Ligase Buffer | 1.0 | μL |
| Target gene DNA | 1.0 | μg |
| Vector pET30a | 1.0 | μg |
| T4 DNA Ligase | 1.0 | μL |
| Ultrapure water | Up to 10 | μL |

1.3.4 Transformation of Ligation Products

(1) The screening medium plate was preheated at 37° C. in advance.

(2) 100 μL of E.coli DH5α competent cells were taken at −80° C., and quickly inserted into ice. After melting, 2.0 μL of ligation products were added, and then mixed gently and bathed in ice for 30 min.

(3) Heat shock in 42° C. water bath for 90s and ice bath for 2 min.

(4) 700 μL LB liquid culture solution preheated at 37° C. was added to each tube, and shake cultured at 37° C. for 60 min at 200 r/min.

(5) 250 μL bacterial solution was evenly coated on the blue-white screening plate (LB agar plate coated with 20 μL 0.1 mM IPTG and 40 μL 20 mg/mL X-gal in advance, dried at 37° C.) of T-A clone containing 100 μg/mL kanamycin, which was uprightly kept at 37° C. until the coating solution was basically absorbed, and then reversely cultured overnight at 37° C.

1.3.5 Plasmid Extraction and Purification

(1) A single well-separated white colony was selected from the plate with an inoculation ring, inoculated into 5 mL LB liquid medium containing 0.1 μg/mL kanamycin, and shake cultured at 160 r/min at 37° C. overnight.

(2) 1.5 mL bacterial solution was added into 2 mL Epperdorf tube, and centrifuged at 5000 r/min at 4° C. for 2 min.

(3) The supernatant was discarded, and 100 μL of solution I precooled by ice bath was added into the tube, the tube was shaken violently and mixed evenly, and treated with ice bath for 5 minutes.

(4) 250 μL Solution II was added into the tube, the tube was mixed upside down for 3-6 times(avoid shaking), and treated with ice bath for 5 minutes.

(5) 150 μL Solution III was added into the tube, the tube was mixed gently upside down for 3-6 times, and treated with ice bath for 5 minutes.

(6) The tube was centrifuged at 4° C. for 10 min at 13000 r/min, the supernatant was carefully sucked and transferred to another 1.5 mL Eppendorf tube, and the tube was added with equal volume of phenol-chloroform (1:1, pH 7.8-8.0), mixed evenly by hand, and placed at room temperature for 2 min.

(7) The tube was centrifuged at 4° C. for 10 min at 13000 r/min, the supernatant was carefully sucked and transferred to another 1.5 mL Eppendorf tube, and the tube was added with equal volume of chloroform, mixed evenly by hand, and placed at room temperature for 2 min.

(8) The tube was centrifuged at 4° C. for 10 min at 13000 r/min, the supernatant was carefully sucked and transferred to another 1.5 mL Eppendorf tube, and the tube was added with 2 times volume of precooled absolute ethanol, mixed evenly by hand, and placed at room temperature for 2 min.

(9) The tube was centrifuged at 10,000 r/min at 4° C. for 10 min, and the supernatant was discarded, the 1.5 mL Eppendorf tube was put upside down on an absorbent paper to suck the residual liquid, and then the precipitate was washed with 1 mL 70% ethanol, centrifuged at 10,000 r/min at 4° C. for 5 min, discarding the supernatant, and volatilized at 37° C.

(10) The precipitate was dissolved in about 30 μL TE buffer and stored at −20° C.

1.3.5 Plasmid Digestion Identification

Enzyme digestion reaction system: 1 μL of restriction endonucleases Nde I and 1 μL of Hind III, 2 μL of 10xenzyme digestion buffer, 10 μL of plasmid (about 1.0 μg), 5.5 μL of ultrapure water, total volume 20 μL, water bath at 37° C. for 2 hours.

10 μL of enzyme digestion product was mixed with the same volume of 2× electrophoresis sample loading buffer, and then added into the sample hole of 1.5% agarose gel containing 1 μg/mL ethidium bromide, and then electrophoresed at 100V for 30min, and the results were observed by ultraviolet detector.

1.3.6 DNA Sequence Determination

The correct cloning of enzyme digestion results was entrusted to Shanghai Introvigen for sequencing.

1.4 Expression and Purification of Target Recombinant Protein

1.4.1 Induced Expression of Target Recombinant Protein

(1) The recombinant expression plasmid verified by sequencing was transformed into E.coli BL21DE3 competent cells, and the method is the same as above.

(2) A positive single colony was selected and inoculated in 10 mL LB liquid medium containing 50 μg/mL kanamycin, and cultured at 200 r/min at 37° C. overnight.

(3) The absorbed culture solution was inoculated in 10 mL LB culture solution in the ratio of 1:100, and was shaken at 37° C. at 200 r/min until the OD 600 was about 0.8.

(4) IPTG with a final concentration of 0.5 mm was added to induce the expression of the target recombinant protein at 37° C. for 4 hours.

(5)1 mL of bacteria solution was centrifuged at 12000 r/min for 1 min, and the bacteria were collected.

(6) The bacteria were washed twice with 0.01 mol/L PBS at a pH of 7.4, and the bacterial precipitate was resuspended with 50 μL PBS.

(7) The bacteria was ultrasonic broken (ultrasonic breaking for 1 min, working time for 3s, interval for 3s) in ice bath.

(8) The bacteria after ultrasonication was centrifuged at 12000 r/min for 10 min, the supernatant was transferred to another test tube, the precipitate was added to 200 μL PBS for resuspension. 200 μL of supernatant and 200 μL of precipitate were added to 4 μL of 5×SDS loading buffer, mixed evenly, bathed in boiling water for 5 min, and detected by SDS-PAGE.

1.4.2 Identification of the Expressed Target Recombinant Protein by SDS-PAGE.

(1) Preparation of 8% separation gel solution: 6.9 mL of double distilled water, 4.0 mL of 30% acrylamide-bisacrylamide, 3.8 mL of Tris(pH 8.8), 0.15 mL of 10% SDS, 0.15 mL of 10% ammonium persulfate, 0.009 mL of TEMED. Separation gel was prepared, the space was left for perfusing stacking gel and sealed with double distilled water.

(2) After standing at room temperature for 30 min, the top of the gel was washed several times with double distilled water to remove unpolymerized acrylamide. The liquid from the gel should be discharged as much as possible, and then the residual liquid was absorbed with the edge of filter paper.

(3) 8 mL of 5% stacking gel was prepared, and the stacking gel solution was perfused on the polymerized separation gel, a clean Teflon stripe was immediately inserted into the concentrated gel solution, and the concentrated gel solution was left to stand at room temperature for 30 min.

(4) After the polymerization of the stacking gel was completed, the stripe was carefully pulled out of gel, and the sample tank was washed with Tris-glycine electrophoresis buffer.

(5) The bacterial solution was centrifuged and precipitated. After PBS was re-suspended, an equal amount of 2×SDS loading buffer was added to mix well. The solution was bathed at 100° C. for 3 min, and about 20 μL of sample was loaded per well.

(6) Turning on the power supply, the constant voltage electrophoresis was carried out at 70V, after the leading edge of bromophenol blue indicator entered the separation gel, the constant voltage electrophoresis was carried out at 150V until the bromophenol blue indicator reached the lower end of the separation gel, and then the power supply was turned off.

(7) Dyeing with dyeing solution for 2 h, then decolorizing with decolorizing solution for 3-5 h.

(8) The gel was removed and stained with Coomassie brilliant blue for 30-60 min.

(9) The gel was transferred to decolorize in decolorizing solution, during which the decolorizing solution can be replaced until the background was clear, and the expression of target recombinant proteins rOmp18, rAhpC and rFlgH was observed.

1.4.3 Purification of Target Recombinant Protein

(1) A single positive colony was selected and inoculated in 10 mL LB liquid culture medium containing 50 μg/mL kanamycin at 200 r/min at 37° C. for overnight culture.

(2) The culture solution was inoculated in 10 mL LB liquid medium contain 50 μg/mL kanamycin in the ratio of 1:100, and cultured at 37° C. for 200 r/min until the OD 600 value was about 0.8.

(3) IPTG with final concentration of 0.5 mM was added to induce the expression of recombinant protein at 37° C. for 4 hours.

(4) The bacterial solution was centrifuged at 12000 r/min for lmin to collect bacterial precipitates.

(5) PBS(pH7.4) buffer solution was used to wash the bacteria twice, and the supernatant was discarded after centrifugation at 12000 r/min for 10 min.

(6) Bacterial precipitate was suspended in 20 mM PB(pH7.2), 300 mM NaCl, 1% Triton X-100, 2 mM DTT, 0.5 mM PMSF solution for ice bath ultrasonic cracking (ultrasound 1 min, working 3s, interval 3s).

(7) 1/20 volume of NTA-0 Buffer and PMSF was added into ice bath. PMSF was prepared into 200 mM storage solution with absolute ethanol and stored at 4° C., with the working concentration of 1 mM.

(8) Lysozyme with final concentration of 0.2-0.4 mg/mL was added into ice bath, mixed uniformly, ice bath for 30 min, and the bacteria was ultrasonically broken.

(9) TritonX-100 with final concentration of 0.05% was added, mixed well, ice bath for 15 min, and the test tube was shaken occasionally to mix well. The cell was ultrasonically broken in ice bath.

(10) MgCl₂ with a final concentration of 5 mM was added and mixed evenly. DNase with final concentration of 10 mg/m1 was added, mixed well, and stood at room temperature for 10 min.

(11) The above solution was centrifuged at 4° C. for 15 min at 5000 r/min, the supernatant was stored it at −20° C.

(12) NTA resin was packed into a suitable chromatographic column, and NTA-0 buffer solution with 10 times NTA volume was passed through the column. The sample was added to NTA chromatographic column, with the flow rate controlled at about 15 mL/h, and the penetrating liquid was collected for SDS/PAGE analysis of protein binding.

(13) Buffer solution with 5 times NTA volume was used to pass through the column, with the flow rate controlled at about 30 mL/h.

(14) The column was eluted with NTA-20, NTA-40, NTA-60, NTA-80, NTA-100 and NTA-500 buffer solution with 5 times NTA volume, with the flow rate controlled at about 15 mL/h, and the eluent was collected.

(15) SDS-PAGE electrophoresis was used to determine the expression of target recombinant proteins rOmp18, rAhpC and rFlgH and their distribution in the eluent.

(16) The eluent containing the target recombinant protein was put in a dialysis bag, the bag was dialyzed at 4° C., during which the dialysate was changed.

(17) After dialysis, the target recombinant protein was concentrated with PEG20000, and then the concentrations of target recombinant protein rOmp18, rAhpC and rFlgH were determined by BCA method, and then stored at −80° C. after subpackage.

1.5 Preparation of rOmp18, rAhpC and rFlgH Antiserum

1.5.1 Animal Immunity

(1) Six healthy male New Zealand rabbits, each weighing about 3 kg, were selected, and about 2 mL of blood was collected from ear vein before immunization. The separated serum was mixed with physiological saline containing 60% glycerol at 1:1 and stored at −70° C.

(2)1 mL containing 2 mg rOmp18, rAhpC or rFlgH, BCG and 1 mL Freund's incomplete adjuvant were fully emulsified in 5 mL syringe.

(3) Primary immunization: New Zealand rabbits were immunized by multiple subcutaneous injections of 2 mL emulsified antigen on the back.

(4) After the first immunization, rabbits were immunized three times at intervals of one week.

(5) 10 days after the last immunization, about 50 mL of heart blood was collected with a 50 mL sterile syringe, which was placed in two 50 mL round-bottom centrifugal tubes, and was placed at 37° C. for 1-2 hours, and after blood coagulation, the tube was placed at 4° C. for 1-2 hours, so that blood clots were fully contracted to separate out serum. The serum was centrifuged at 3000 r/min, 4° C. for 5 min, and stored at −20° C.

1.5.2 Purification of IgG

(1) the solution was filtered with a membrane with a pore size of 0.45 μm or 0.2 μm.

(2) All solutions must be degassed by ultrasonic methods.

(3) An appropriate purification column was selected and filled with an appropriate amount of Protein A Agarose.

(4) The purification column was washed and balanced with TBS with 10-20 times of column volume.

(5) The sample containing the antibody to be purified was loaded to the purification column.

(6) After the antibody to be purified passed through the column, the column was washed with TBS with 10-20 times of column volume to remove the unbound and nonspecific binding proteins. Completion of the washing can be determined by measuring the absorbance at 280 nm.

(7) The bound IgG was eluted with 10 mL 50 mM glycine(pH2.7), the eluted antibody was collected separately, and the collection tube where the IgG elution peak is located was determined according to the detection result of protein concentration.

(8) The antibody eluent was collected and dialyzed in PBS at 4° C. overnight.

1.5.3 Identification of IgG

1.5.3.1 Determination of IgG Concentration

The protein concentration of IgG extract was determined by Bradford method, and the detection steps referred to the instructions of Bradford protein concentration determination kit of Shanghai Beyotime Biotechnology Co., Ltd.

1.5.3.2 Determination of Antibody Titer by ELISA

(1) Coating: rOmp18, rAhpC and rFlgH were prepared into 5 μg/mL solution with coating buffer, and then added into the plate, with 100 μL per hole, at 4° C. overnight.

(2) Sealing: the coating solution was discarded, and the plate was washed with TBST for 3 times, 200 μL sealing solution was added to each well, and incubated at 37° C. for 1 h, the sealing solution was discarded, and the plate was washed with TBST for 2 times.

(3) Primary antibody reaction: rOmp18-IgG, rAhpC-IgG or rFlgH-IgG were diluted at 1:500, 100 μL was added to each well, and incubated at 37° C. for 1 hour.

(4) Secondary antibody reaction: the primary antibody solution was discarded, the plate was washed with TBST for 3 times, 100 μL of HRP labeled goat anti-rabbit secondary antibody in 1:5000 dilution was added to each well, and incubated at 37° C. for 1 hour.

(5) Color development: the secondary antibody solution was discarded, the plate was washed with TBST for 4 times, 100 μL TMB color development solution was added to each hole, and the color development time was determined according to the color depth, usually at 37° C. for 15 min.

(6) Termination of the reaction: 100 μL 1 M HCl solution was added to each well to terminate the reaction, the OD value at 450 nm was immediately detected on the enzyme-labeled instrument, and the dilution degree corresponding to the well whose OD 450 value is 2.1 times higher than the set negative control OD 450 value was determined as the titer of the antibody.

1.6 Dot Blot and ELISA were used to Detect Dominant Epitopes

1.6.1 Dot Blot Detection

(1) Sample addition: 5 μL of T-B combined epitope peptide of Omp18, AhpC or FlgH with the same concentration was dripped on PVDF membrane, and naturally dried.

(2) Sealing: the PVDF membrane was immersed in a sealing solution containing 5% skimmed milk powder, slowly shaken on a shaker, and sealed at room temperature for 1 h.

(3) Membrane washing: the membrane was washed with 1×TBST on the shaking table for 10 min, and the washing solution was changed for 3 times.

(4) Primary antibody: 1:500 diluted anti-rOmp18-IgG, rAhpC-IgG or rFlgH-IgG was added as primary antibody, and PVDF membrane was incubated on shaking table at room temperature for 1 h.

(5) Membrane washing: the membrane was washed with 1×TBST for 10 min, and the solution in shaking table was changed for 3 times.

(6) Effect of secondary antibody: 1:4000 diluted HRP-labeled goat anti-rabbit IgG secondary antibody was added, and PVDF membrane was incubated on shaking table at room temperature for 1 h.

(7) Membrane washing: the membrane was washed with 1×TBST for 10 min, and the solution in shaking table was changed for 3 times.

(8) ECL chemiluminescence development: the PVDF film was placed on the preservative film, and a proper amount of solution A and solution B with equal volume in ECL kit were mixed, then added on the surface of the film after being evenly mixed, and then transferred to a gel imaging analyzer for exposure development in chemical photosensitive mode.

1.6.2 Detection of the Immunoreactivity between T-B Combined Epitope Peptide and Antibody by ELISA

The synthetic Omp18, AhpC or FlgH different T-B combined epitope peptides were prepared into 1 g/mL solution with coating buffer respectively, and 50 pL was added to each reaction well for overnight at 4° C. On the next day, the solution in the well was discarded, the plate was washed once by TBST, sealed at 37° C. for 1 h with 1% BSA, and then 1:200, 1:1000, 1:5000, 1:10000, 1:20000, 1:60000 or 1:240000 diluted rOmp18-IgG, rAhpC-IgG or rFlgH-IgG were added as primary antibody, the plate was oscillating incubated at room temperature for lh, then washed with TBST for three times; 1:10000 diluted HRP labeled goat anti-rabbit IgG secondary antibody was added, incubated at 37° C. for 45 min, the plate was washed with TBST for 3 times, substrate solution was added to develop color, after terminating the reaction, the enzyme-labeled instrument was used to read the information from the plate.

1.7 Construction and Identification of the Tandem Polypeptide Gene of Omp18, AhpC and FlgH Dominant T-B combined Epitope

According to the results of Dot Blot and ELISA, three dominant T-B combined epitopes were selected and named Omp18-1, AhpC-2 and FlgH-1 respectively.

According to the literature (Wei JC, et al. biochem biophy RES commun, 2010), the dominant T-B combined epitope peptide sequences of AhpC-2, Omp18-1 and FlgH-1 were connected with GGS sequences to construct the triple repeat tandem gene of the trivalent dominant T-B combined epitope peptide. According to the codon optimization software MaxCodon™ Optimization Program (V13), the amino acid codons of repeated tandem were optimized, and the artificial gene fragment of triple repeated tandem of trivalent dominant T-B combined epitope peptide was synthesized by whole gene synthesis method. The gene was inserted into prokaryotic expression vector pET30a through restriction endonuclease sites Nde I and Hind III. The correctness of the recombinant expression vector sequence was confirmed by double digestion and sequencing, and then the recombinant expression vector was transformed into Escherichia coli Top10 clone strain and BL21DE3 expression strain, respectively, and the triple repeat tandem prokaryotic expression system of trivalent dominant T-B combined epitope peptide was constructed. The recombinant prokaryotic expression vector pET30a is shown in FIG. 1.

1.8 Expression and Purification of rAhpC-2/Omp18-1/FlgH-1

The expression and purification methods of target recombinant protein rAhpC-2/Omp18-1/FlgH-1 are the same as 1.4.1 and 1.4.3.

1.9 Detection of Immunogenicity of rAhpC-2/Omp18-1/FlgH-1

1.9.1 Animals Immunized with rAhpC-2/Omp18-1/FlgH-1

90 SPF female BALB/c mice aged 4-6 weeks were randomly divided into 5 groups, namely, group A (25 μg rAhpC-2/Omp18-1/FlgH-1+ complete Freund's adjuvant), group B (25 μg rAhpC+ complete Freund's adjuvant), group C (25 μg rOmp18+ complete Freund's adjuvant), group D (25 μg rFlgH+complete Freund's adjuvant), and group F (equivalent NS+complete Freund's adjuvant, control group). Immunization method is the same as 1.5.1.

1.9.2 Detection of Serum Specific Antibodies IgG1 and IgG2a by ELISA

(1) Five mice were randomly selected from each group, and blood was collected 2 weeks after the last immunization, and serum IgG was determined by ELISA.

(2) Coating: 5 μg/mL purified rAhpC-2/Omp18-1/FlgH-1, rAhpC, rOmp18 and rFlgH solution was prepared with coating buffer, 100 μL per well, overnight at 4° C.

(3) Washing: the plate was washed with 1×PBST for 3 times.

(4) Sealing: 100 μL of 5% BSA-PBST was added to each well and sealed at 37° C. for 1 hour.

(5) Washing: the plate was washed with 1×PBST for 3 times.

(6) Primary antibody: 1:100 diluted rabbit antisera of rAhpC-2/Omp18-1/FlgH-1, rAhpC, rOmp18 and rFlgH was added to each well, and incubated at 37° C. for 2 h.

(7) Washing: the plate was washed with 1×PBST for 3 times.

(8) Secondary antibody: 100 μL 1:10000 diluted HRP labeled sheep anti-rabbit IgG1 or IgG2a was added to each well, and incubated at 37° C. for 1 h.

(9) Color development: 100 μL TMB substrate color development solution was added to each well, and incubated at 37° C. for 15min in the dark.

(10) Terminating the reaction: 100 μL 2M sulfuric acid was added to each hole to terminate the reaction.

(11) Determination of results: OD 450 value was detected by enzyme-labeled instrument at 450 nm wavelength.

1.9.3 Detection of Serum Specific antibodies by Western Blot

(1) Equal amount of rAhpC-2/Omp18-1/FlgH-1, rAhpC, rOmp18, rFlgH SDS-PAGE was semi-dry electric transferred to PVDF membrane at 23V for 45 min.

(2) PVDF membrane was dyed with Lichun red dye solution for 1 min, the membrane transfer effect was checked, then the membrane was rinsed with double distilled water, and sealed with 5% FCS-TBST overnight at 4° C.

(3) The antiserum diluted with TBST 1:1000 was used as primary antibody, and PVDF membrane was incubated at room temperature for 2 h at 90 r/min, rinsed with TBST for 30 min, and the solution was changed every 5 min.

(4) The IgG labeled with sheep anti-rabbit HRP diluted by TBST 1:5000 was used as the second antibody, and PVDF membrane was incubated at room temperature for 1 hour at 90 r/min.

(5) The membrane was rinsed with TBST for 10 min, and the solution was changeds every 5 min.

(6) The PVDF membrane was taken out and drained carefully on absorbent paper, and the membrane was incubated with ECL (5 mL of solution A and solution B and 20 μL of hydrogen peroxide were mixed evenly) for 1 min.

(7) The PVDF film was drained, and spread on the preservative film, another layer of preservative film was covered, the bubbles were driven out, and the membrane was placed in the exposure clip. Turning off all lights, the exposure time was estimated according to the fluorescence intensity, the film was put into developer and fixer for 7 min after exposure respectively, rinsed with clear water and dried, and the results was observed.

1.10 Detection of Splenic Lymphocyte Subsets by Flow Cytometry

1.10.1 Cell Separation

(1) After weighing the spleen tissue of mice, the spleen was cut into small pieces by aseptic operation with ophthalmic scissors.

(2) The spleen tissue mass was put on a 70 μm cell screen, ground repeatedly, and then homogenate washing solution (take 0.1 g tissue as an example, add about 5-8 ml) was added while grinding, so that the cells can drip into the centrifuge tube through the screen.

(3) The screen was discarded, the spleen tissue grinding solution was centrifuged at 450 r/min for 10 min, and the supernatant was discarded.

(4) Cell precipitation was resuspended with sample diluent and counted, and the cell concentration of cell suspension was adjusted to 2×10⁸−1×10⁹/mL for later use.

(5) The same amount of separation solution (the separation solution shall not be less than 4 mL) as the splenic single cell suspension was added into a suitable centrifuge tube.

(6) The spleen single cell suspension was carefully sucked and added to the solution surface of the separation solution, and centrifuged for 30 min at 400-500 r/min.

(7) After centrifugation, the solution in the centrifuge tube was divided into four layers from top to bottom: the first layer was diluent layer, the second layer was annular milky white lymphocyte layer (containing a small amount of red blood cells), the third layer was transparent separation solution layer, and the fourth layer was red blood cell layer.

(8) The second annular milky white lymphocyte layer was carefully sucked into another 15 mL centrifuge tube, 5-10 mL washing solution was added into the centrifuge tube, and mixed well.

(9) The cell suspension was centrifuged at 400 r/min for 10 min. The supernatant was discarded, and the cell pellet was resuspended with 5 mL washing solution.

(10) The cell suspension was centrifuged at 250 r/min for 10 min.

(11) Steps (9) and (10) were repeated to obtain spleen lymphocytes needed for subsequent experiments.

1.10.2 Detection of Splenic Lymphocyte Subsets by Flow Cytometry

Spleen lymphocyte suspension was filled into flow tubes, each tube was added with 100 μL CD4-IgG, CD8-IgG, IL4-IgG and IFN-γ-IgG, and incubated at room temperature for 30 min in the dark. The different T cell subsets in spleen lymphocytes were detected by flow cytometry, and the CD4+cells, IFN-γ+CD4+cells, IL4+CD8+cells of spleen lymphocytes of mice in different experimental groups were known.

1.11 Animal Protection Test

1.11.1 Animal Immunity

BALB/c mice were randomly divided into five groups: rAhpC-2/Omp18-1/FlgH-1 group, rOmp18 group, rAhpC group, rFlgH group and normal control group, with 13 mice in each group. Each mouse in the first four groups was immunized subcutaneously with 25 μg corresponding recombinant protein and Freund's complete adjuvant at intervals of one week, for four times.

1.11.2 Campylobacter jejuni Attack

On the 1st, 3rd and 7th day after the last immunization, 1 mL Campylobacter jejuni (5 x10 9 CFU) was given to the stomach, and then the morbidity and mortality of animals within 8 weeks were observed every day and the disease index of each group was calculated. Scoring criteria: 0 for physical health, 1 for symptoms and signs, and 2 for death. Mice with one or more of obviously decreased activity, decreased appetite, hunched back, scattered and gray fur, and wilting spirit are considered to have symptoms and signs. The sum of disease indexes of each group is divided by the number of animals observed on that day to obtain the disease index of this group, and then the immune protection rate is calculated according to the following formula: protection rate=(control group disease index—immune group disease index)/control group disease index×100%.

1.11.3 Sample Collection of Mouse Jejunum

Two weeks after Campylobacter jejuni attack, the mice were killed by cervical dislocation, the abdomen was opened, 2 cm jejunum was taken, the contents were collected and suspended in sterilized PBS, and another 2 cm jejunum was taken and fixed with 4% PFA.

1.11.4 Evaluation of Immune Protection

After HE staining, the intestinal tissues of mice were examined by ordinary optical microscope. Intestinal mucosa without injury but with slight inflammatory cell infiltration is classified as mild inflammation, intestinal mucosa with moderate inflammatory exudate and partial necrosis is classified as moderate inflammation, and intestinal mucosa is almost completely stripped and a large number of inflammatory cell infiltration, inflammatory exudate and necrosis are classified as severe inflammation.

2 Data Processing and Statistical Analysis

The experimental data are expressed as mean±standard deviation, and the statistical software GraphPad Prism was used to analyze the mean of multiple groups. First, the data were tested for homogeneity of variance. If the variance is homogeneous, single factor analysis of variance was used for overall comparison, LSD method was used for statistical analysis between the mean of each experimental group and the control group, and rank sum test was used for statistical analysis of data with abnormal or uneven variance.

3 Results and Analysis

3.1 Prediction of Outer Membrane Protein T-B Combined Epitope of

Campylobacter jejuni

The linear B cell epitopes of Omp18, AhpC and FlgH of Campylobacter jejuni NCTC11168 strain were predicted by IEDB online prediction software, and the T cell epitopes were predicted by Epitope Prediction online software. According to the scores of T cell and T cell epitopes and their overlap (T-B combined epitopes), six high-score T-B combined epitopes were obtained (table 2).

TABLE 2
Prediction results of outer membrane protein T-B combined epitope of
Campylobacter jejuni

| Epitope | Position | Sequence (N-C) |
|---|---|---|
| AhpC | 110-132 | 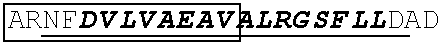 |
| | | As set forth in SEQ ID NO: 6 |
| AhpC-2 | 4-39 | 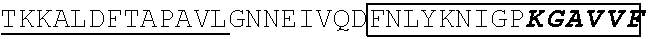 |
| | | As set forth in SEQ ID NO: 5 |
| Omp18-1 | 117-158 | 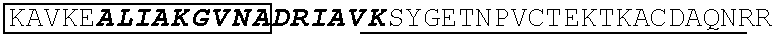 |
| | | As set forth in SEQ ID NO: 4 |
| Omp18-2 | 3-47 | 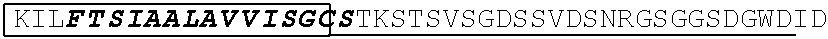 |
| | | As set forth in SEQ ID NO: 7 |
| FlgH-1 | 14-52 | 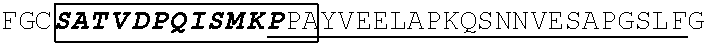 |
| | | As set forth in SEQ ID NO: 3 |
| FlgH-2 | 64-97 | 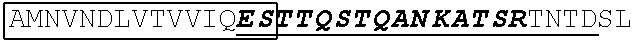 |
| | | As set forth in SEQ ID NO: 8 |

Wherein B cell epitopes are underlined, human T cell epitopes are in boxes, and mouse T epitopes are bold and italicized.

3.2 Identification of Recombinant Expression Vector of Campylobacter jejuni Outer Membrane Protein by Enzyme Digestion

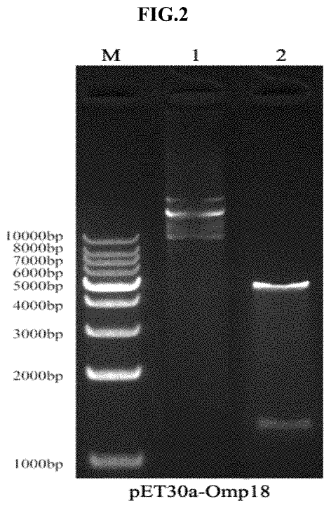

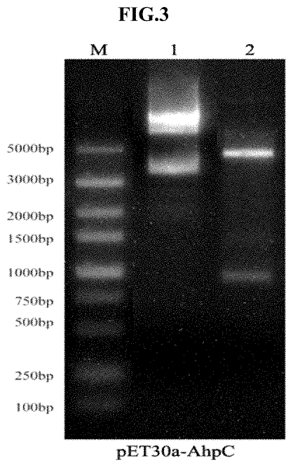

The results of Nde I and Hind III digestion after linking Omp18, AhpC and FlgH genes of Campylobacter jejuni with pET30a are shown in FIG. 2-FIG. 4 (Lane M is DNA marker; lane 1 is recombinant pET30a; lane 2 is the result of Nde I and Hind III digestion). FIG. 2 shows the double digestion results of Nde I and Hind III after connecting Omp18 gene of Campylobacter jejuni with pET30a; FIG. 3 shows the double digestion results of Nde I and Hind III after connecting AhpC gene of Campylobacter jejuni with pET30a; FIG. 4 shows the results of Nde I and Hind III digestion after connecting FlgH gene of Campylobacter jejuni with pET30a.

3.3 Expression, Purification and Immunogenicity Detection of Campylobacter jejuni Recombinant Protein

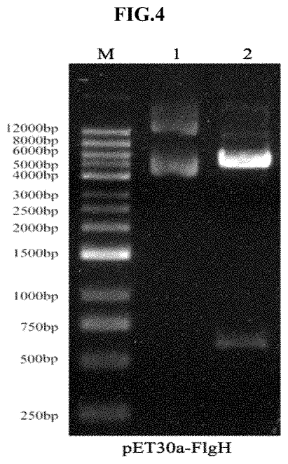

The constructed prokaryotic expression system of Campylobacter jejuni Omp18, AhpC and FlgH genes can effectively express the target recombinant proteins rOmp18, rAhpC and rFlgH induced by IPTG. SDS-PAGE test result showed that rOmp18 (about 17kda), rAhpC (about 22 kda) and rFlgH (about 24 kda) purified by Ni-NTA affinity chromatography showed a single protein band(A in FIG. 5). The purified rOmp18, rAhpC and FlgH concentrations purified by Bradford method were 0.745 mg/mL, 1.150 mg/mL and 0.381 mg/mL, respectively. The results of Western Blot showed that rOmp18, rAhpC and rFlgH could have specific immune binding reaction with corresponding antibodies (B in FIG. 5).

3.4 Screening of Dominant T-B combined epitope of Campylobacter jejuni Omp18, AhpC and FlgH

Dot Blot test results show that in the two T-B combined epitopes of Campylobacter jejuni Omp18, AhpC and FlgH, rOmp18-IgG can bind to Omp18-1 and Omp18-2 epitopes, but the former has stronger binding reaction than the latter, rAhpC -IgGAhpC and rFlgH-IgG can show strong binding reactions only with AhpC-2 and FlgH-1, respectively (FIG. 6), suggesting that Omp18-1, AhpC-2 and FlgH-1 are the dominant TB combined epitopes.

3.5 Identification of Tandem Epitope Peptide of Campylobacter jejuni AhpC-2/Omp18-1/FlgH-2

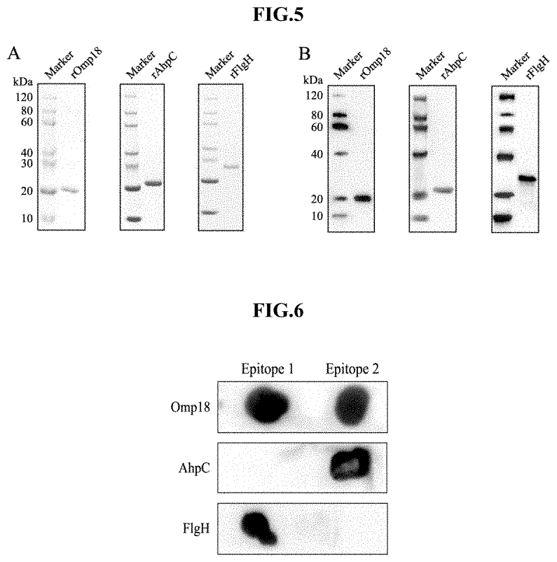

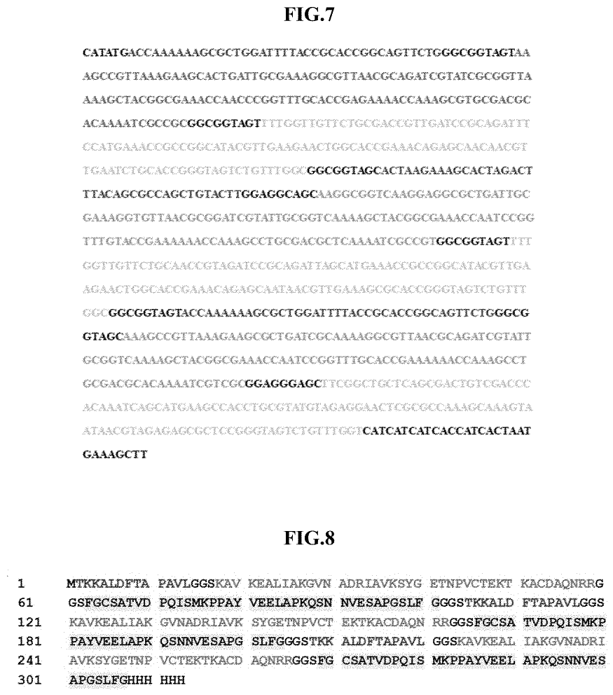

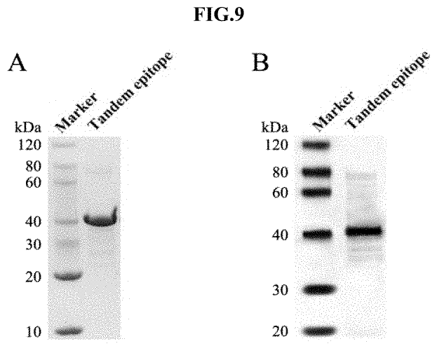

Sequencing results of recombinant pET30a of rAhpC-2/Omp18-1/FlgH-1 are shown in FIG. 7. The amino acid sequence is shown in FIG. 8. SDS-PAGE shows that IPTG can induce tandem repeat prokaryotic expression system of rAhpC-2/Omp18-1/FlgH-1 to express the trivalent recombinant T-B combined epitope peptide rAhpC-2/Omp18-1/FlgH-1 (A in FIG. 9). The results of Western Blot show that rAhpC-2/Omp18-1/FlgH-1 can have strong specific immune binding reaction with the whole antibody of Campylobacter jejuni (B in FIG. 9).

3.6 Changes of Immune Indexes after Immunizing Animals with Tandem T-B Combined Epitope Peptide

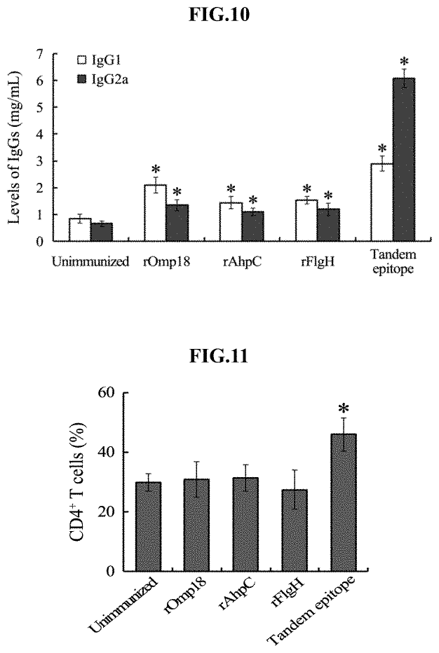

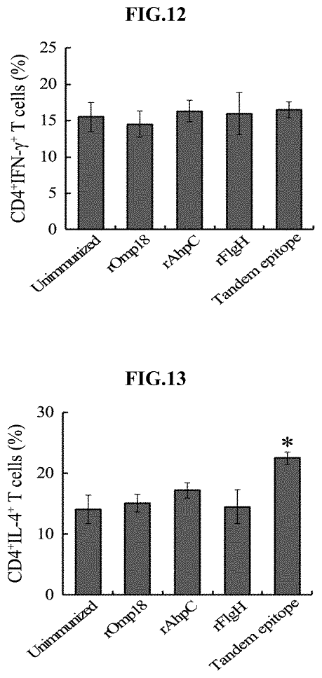

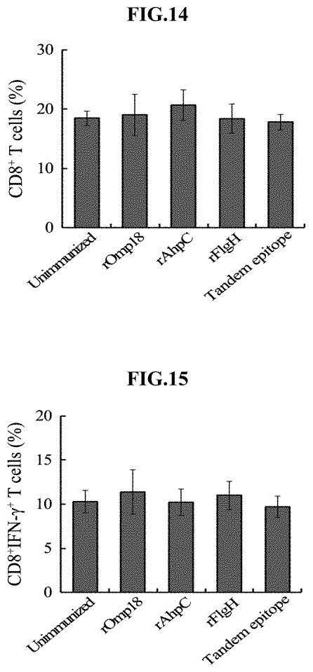

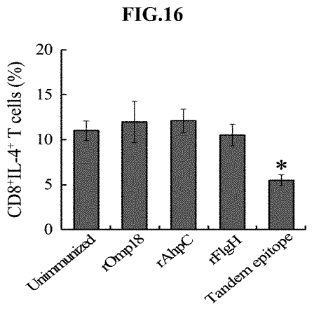

ELISA results show that serum IgG antibody levels of mice immunized with rAhpC, romp 18 and rFlgH all increase (P<0.05), but the serum IgG level of mice immunized with T-B combined rAhpC-2/Omp18-1/FlgH-1 tandem epitope peptide increases more significantly (P<0.05), and the main types are IgG1 and IgG2a (FIG. 10) The results of flow cytometry show that the number of CD4+ cells in spleen lymphocytes immunized with rOmp18, rAhpC and rFlgH doses not change significantly (P>0.05), but the number of CD4+ cells in spleen lymphocytes immunized with rAhpC-2/Omp18-1/FlgH-1 tandem epitope peptide increases significantly (P<0.05), wherein CD4+IL-4+ cells increase significantly, and the number of CD4 +IFN-γ+ cells does not change significantly (P>0.05). CD8+ cells and IFN-γ+CD8+ cells in spleen lymphocytes immunized with rAhpC, rOmp18, rFlgH and rAhpC-2/Omp18-1/FlgH-1 tandem epitope peptide do not change significantly (P>0.05). However, the number of CD8+IL-4+ cells in mice immunized with rAhpC-2/Omp18-1/FlgH-1 tandem epitope peptide decreases significantly (P<0.05) (FIG. 11-FIG. 16, *indicates P<0.05 compared with non-immunized mice, wherein FIG. 11 shows the change of CD4+cells; FIG. 12 shows the change of CD4+IFN-γ+ cells. FIG. 13 shows the change of CD4+IL-4+ cells. FIG. 14 shows the change of CD8+ cells. FIG. 15 shows the change of IFN-γ+CD8+ cells. FIG. 16 shows the change of CD8+IL-4+ cells.

3.7 Immunoprotective Effect of Tandem T-B combined Epitope Peptide

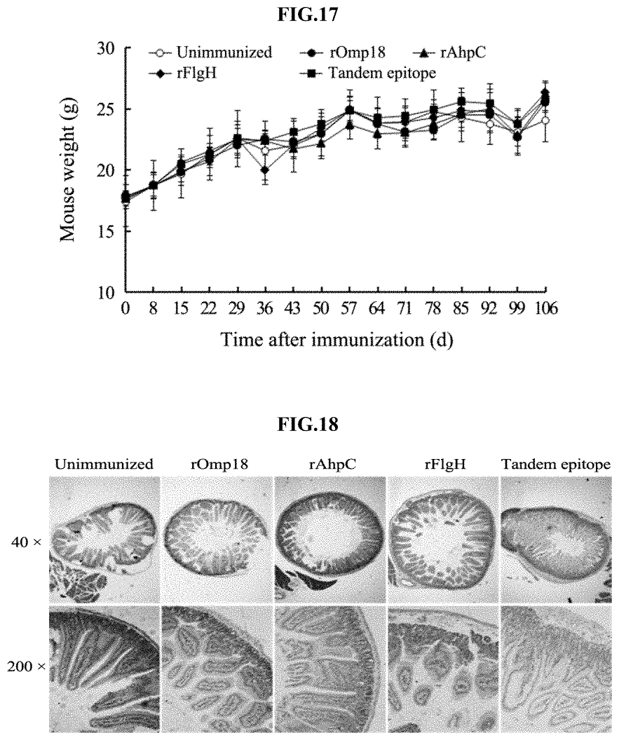

The results of animal experiments show that the positive rate of Campylobacter jejuni in the contents of the jejunum is 90% and the number of colonies could reach 10⁴ CFU/mL after the infection of Campylobacter jejuni via the digestive tract in the unimmunized control group. The positive rate of Campylobacter jejuni in the jejunum content of mice immunized with rOmp18, rAhpC, rFlgH, rAhpC-2/Omp18-1/FlgH-1 tandem TB combined epitope peptides is significantly reduced (P<0.05), wherein the positive rate of Campylobacter jejuni in mice immunized with rAhpC-2/Omp18-1/FlgH-1 is reduced by 40%, which is significantly lower than that in mice immunized with rOmp18, rAhpC, and rFlgH (P<0.05). The results of disease index analysis show that the disease index of mice immunized with rAhpC-2/Omp18-1/FlgH-1 is significantly lower than that of mice immunized with rOmp18, rAhpC, and rFlgH-1 (P<0.05), and the protection rate can reach 80% (Table 3). After immunization, the body weight of mice in each group increases without significant difference (FIG. 17). Pathological examination results show that rOmp18, rAhpC and rFlgH immunized mice have more inflammatory cell infiltration in the intestinal mucosa, rAhpC-2/Omp18-1/FlgH-1 immunized mice have intact intestinal mucosa without obvious inflammatory cell infiltration (FIG. 18).

TABLE 3
Disease index and protection rate of immunized
mice infected with Campylobacter jejuni

| | | Protection |
|---|---|---|
| Groups | Disease index | rate (%) |
| rAhpC-2/rOmp18-1/rFlgH-1 | 0.20 ± 0.42^{*#} | 80 |
| rAhpC | 0.50 ± 0.71^{*} | 50 |
| rOmp18 | 0.60 ± 0.52^{*} | 40 |
| rFlgH | 0.70 ± 0.67^{*} | 30 |
| Unimmunized control group | 1.00 ± 0.47^{*} | 10 |

The above are only the preferred embodiments of the present disclosure. It should be pointed out that for those of ordinary skill in the art, without departing from the principle of the present disclosure, several improvements and modifications can be made, and these improvements and modifications are also It should be regarded as the protection scope of the present disclosure.

1. A tandem epitope polypeptide of outer membrane protein of campylobacter jejuni, wherein the tandem epitope polypeptide comprises the amino acid sequence of SEQ ID NO: 1.

2. A gene encoding the tandem epitope polypeptide of outer membrane protein of campylobacter jejuni according to claim 1, wherein the gene comprises the nucleotide sequence of SEQ ID NO: 2.

3. A recombinant plasmid comprising the gene of claim 2.

4. The recombinant plasmid according to claim 3, wherein the recombinant plasmid is obtained by inserting the gene into pET30a as an original plasmid.

5. The recombinant plasmid according to claim 4, wherein the gene is inserted between Nde I and Hind III restriction sites on pET30a.

6. A recombinant bacteria comprising the recombinant plasmid according to claim 3.

7. A method for preventing Campylobacter jejuni infection, comprising administering a vaccine to a subject in need thereof, wherein the vaccine comprises the tandem epitope polypeptide of outer membrane protein of campylobacter jejuni according to claim 1.

8. The recombinant bacteria according to claim 6, wherein the recombinant plasmid is obtained by inserting the gene into pET30a as an original plasmid.

9. The recombinant bacteria according to claim 8, wherein the gene is inserted between Nde I and Hind III restriction sites on pET30a.

10. A method for preventing Campylobacter jejuni infection, comprising administering a vaccine to a subject in need thereof, wherein the vaccine comprises the gene according to claim 2.

11. A method for preventing Campylobacter jejuni infection, comprising administering a vaccine to a subject in need thereof, wherein the vaccine comprises the recombinant plasmid according to claim 3.

12. A method for preventing Campylobacter jejuni infection, comprising administering a vaccine to a subject in need thereof, wherein the vaccine comprises the recombinant plasmid according to claim 4.

13. A method for preventing Campylobacter jejuni infection, comprising administering a vaccine to a subject in need thereof, wherein the vaccine comprises the recombinant plasmid according to claim 5.

14. A method for preventing Campylobacter jejuni infection, comprising administering a vaccine to a subject in need thereof, wherein the vaccine comprises the recombinant bacteria according to claim 6.


SEQUENCE LISTING (8 sequences)

SEQ ID NO: 1 - protein, 313 aa, Artificial Sequence
  tandem epitope polypeptide of outer membrane protein of Campylobacter jejuni
MTKKALDFTAPAVLGGSKAVKEALIAKGVNADRIAVKSYGETNPVCTEKTKACDAQNRRGGSFGCSATVDPQISMKPPAYVEELAPKQSNNVESAPGSLFGGGSTKKALDFTAPAVLGGSKAVKEALIAKGVNADRIAVKSYGETNPVCTEKTKACDAQNRRGGSFGCSATVDPQISMKPPAYVEELAPKQSNNVESAPGSLFGGGSTKKALDFTAPAVLGGSKAVKEALIAKGVNADRIAVKSYGETNPVCTEKTKACDAQNRRGGSFGCSATVDPQISMKPPAYVEELAPKQSNNVESAPGSLFGHHHHHH

SEQ ID NO: 2 - DNA, 954 bp, Artificial Sequence
  gene encoding tandem epitope polypeptide of outer membrane protein of Campylobacter jejuni
catatgaccaaaaaagcgctggattttaccgcaccggcagttctgggcggtagtaaagccgttaaagaagcactgattgcgaaaggcgttaacgcagatcgtatcgcggttaaaagctacggcgaaaccaacccggtttgcaccgagaaaaccaaagcgtgcgacgcacaaaatcgccgcggcggtagttttggttgttctgcgaccgttgatccgcagatttccatgaaaccgccggcatacgttgaagaactggcaccgaaacagagcaacaacgttgaatctgcaccgggtagtctgtttggcggcggtagcactaagaaagcactagactttacagcgccagctgtacttggaggcagcaaggcggtcaaggaggcgctgattgcgaaaggtgttaacgcggatcgtattgcggtcaaaagctacggcgaaaccaatccggtttgtaccgaaaaaaccaaagcctgcgacgctcaaaatcgccgtggcggtagttttggttgttctgcaaccgtagatccgcagattagcatgaaaccgccggcatacgttgaagaactggcaccgaaacagagcaataacgttgaaagcgcaccgggtagtctgtttggcggcggtagtaccaaaaaagcgctggattttaccgcaccggcagttctgggcggtagcaaagccgttaaagaagcgctgatcgcaaaaggcgttaacgcagatcgtattgcggtcaaaagctacggcgaaaccaatccggtttgcaccgaaaaaaccaaagcctgcgacgcacaaaatcgtcgcggagggagcttcggctgctcagcgactgtcgacccacaaatcagcatgaagccacctgcgtatgtagaggaactcgcgccaaagcaaagtaataacgtagagagcgctccgggtagtctgtttggtcatcatcatcaccatcactaatgaaagctt

SEQ ID NO: 3 - protein, 39 aa, Artificial Sequence
  epitope FlgH-1
FGCSATVDPQISMKPPAYVEELAPKQSNNVESAPGSLFG

SEQ ID NO: 4 - protein, 42 aa, Artificial Sequence
  epitope OMP18-1
KAVKEALIAKGVNADRIAVKSYGETNPVCTEKTKACDAQNRR

SEQ ID NO: 5 - protein, 36 aa, Artificial Sequence
  epitope AhpC-2
TKKALDFTAPAVLGNNEIVQDFNLYKNIGPKGAVVF

SEQ ID NO: 6 - protein, 23 aa, Artificial Sequence
  epitope AhpC
ARNFDVLVAEAVALRGSFLLDAD

SEQ ID NO: 7 - protein, 45 aa, Artificial Sequence
  epitope Omp18-2
KILFTSIAALAVVISGCSTKSTSVSGDSSVDSNRGSGGSDGWDID

SEQ ID NO: 8 - protein, 34 aa, Artificial Sequence
  epitope FlgH-2
AMNVNDLVTVVIQESTTQSTQANKATSRTNTDSL